(19) United States
(12) Patent Application Publication
(10) Pub. No.: US 2012/0046544 A1
(43) Pub. Date: Feb. 23, 2012

(54) RADIATION TOMOGRAPHY APPARATUS

(75) Inventor: Yoshihiro Inoue, Kyoto-fu (JP)

(73) Assignee: SHIMADZU CORPORATION, Kyoto (JP)

(21) Appl. No.: 13/263,912

(22) PCT Filed: Apr. 16, 2009
(86) PCT No.: PCT/JP2009/001765

§ 371 Date: Oct. 11, 2011

(51) Int. Cl. A61B 5/05

(52) U.S. Cl. 600/425

(57) ABSTRACT

This disclosure has one object to provide radiation tomography apparatus that allows acquisition of a sectional image having an accurately mapped radiopharmaceutical localization in an internal structure of a subject. According to one embodiment, a top board stops several times between a starting position forwardly in a top board movement direction and a termination position backwardly in the top board movement direction. Both CT image and the PET image are acquired upon stop of the top board. With the conventional method, the subject in the PET image and that in the CT image deviate from each other in position. In contrast to this, with the configuration of this disclosure, assumed that once movement of the top board is one step, a CT image taken two steps before has been acquired in every taking of the PET image by several times. Superimposing of these images may realize accurate mapping of radiopharmaceutical localization in the internal structure of the subject.

### TECHNICAL FIELD

This invention relates to radiation tomography apparatus with a PET device for imaging distribution of radiopharmaceutical administered by injection to a subject. Particularly, this invention relates to radiation tomography apparatus provided with a CT device, in addition to the PET device, for acquiring a fluoroscopic image of the subject through irradiating the subject with radiation from outside, thereby acquiring a constrictive sectional image of the subject.

### BACKGROUND ART

Medical institutions are equipped with radiation tomography apparatus that allows imaging of radiopharmaceutical distribution. This type of tomography apparatus detects annihilation radiation (such as gamma rays) emitted from radiopharmaceutical that is administered to a subject M and is localized to a site of interest, and acquires sectional images of the site of interest in the subject M that show radiopharmaceutical distribution.

Now, description will be given of conventional radiation tomography apparatus. Radiation tomography apparatus 50 includes a PET device 50a and a CT device 50b. The PET device 50a detects annihilation radiation. The CT device 50b acquires a fluoroscopic imaging of a subject M. The PET device 50a may obtain information only on radiopharmaceutical distribution. Accordingly, the CT device 50b acquires images on an internal structure of the subject M. The CT device 50b acquires a sectional image having an image of organs in the subject M falling thereon, whereas the PET device 50a acquires a sectional image showing pharmaceutical distribution. Superimposing of these images may realize mapping of radiopharmaceutical localization in the internal structure of the subject M. Here, a sectional image acquired with the CT device 50b is referred to as a CT image, and a sectional image acquired with the PET device 50a is referred to as a PET image.

Description will be given of a configuration of the radiation tomography apparatus 50. As shown in FIG. 12, the radiation tomography apparatus 50 includes a top board 52 for supporting the subject M. Moreover, the radiation tomography apparatus 50 includes a PET device 50a and a CT device 50b in a ring shape each having a hole for inserting the top board 52. See, for example, Patent Literature 1. The top board 52 is slidable along a longitudinal direction thereof (i.e., a z-direction: a direction where the top board 52 passes through the PET device 50a and the CT device 50b.) The PET device 50a includes a detector ring 52 in a ring shape haying a hole extending in the z-direction. The CT device 50b includes a radiation source 53 that rotates around the top board 52 with no variation in positions in the z-direction, and a radiation detector 54 for detecting radiation emitted from the radiation source 53. The radiation source 53 and the radiation detector 54 synchronously rotate along a path in a ring shape provided inside the CT device 50b so as no to change a relative position therebetween.

Next, description will be given of a conventional operation of the radiation tomography apparatus 50. In the conventional radiation tomography apparatus 50, a CT image on a total body of the subject M is firstly acquired for determination of a pharmaceutical distribution inside of the subject M. In this step, only the CT device 50b operates, and the PET device 50a detects no annihilation radiation. That is because the CT device 50b takes a CT image while the radiation source 53 emits radiation and radiation may enter into the PET device 50a. Such radiation from outside of the subject M is obstructive to acquisition of a PET image. Accordingly, the conventional apparatus has no configuration in which both sectional images are simultaneously acquired.

The top board 52 operates prior to acquisition of a CT image to move a head of the subject M between the radiation source 53 and the radiation detector 54. Then, the radiation source 53 rotates while intermittently irradiating the subject M with radiation to continuously taking a fluoroscopic image having a fluoroscopic image of the subject M falling thereon. The top board 52 moves successively during the continuous imaging. Taking CT images is to be completed after taking an image of a tiptoe of the subject M. A series of fluoroscopic images is converted into a CT image through a general back projection method, etc. En this way, a CT image of a total body of the subject M is to be taken at one time.

Subsequently, acquisition of a PET image is performed. The top board 52 operates prior to this imaging to move a head of the subject M into a position where the detector ring 62 covers. The detector ring 62 detects an annihilation gamma-rays pair that is emitted from the head of the subject M. The top board 52 slides after taking a head image of the subject M. Next, the top board 52 moves the subject M into a position where the detector ring covers a chest of the subject M. The detector ring 62 detects an annihilation gamma-rays pair that is emitted from the chest of the subject M. As above, the top board 52 moves in a stepwise fashion, thereby changing a relative position between the detector ring 62 and the subject M. Whenever the position is changed, each site of the subject M is successively introduced into a field of view of the detector ring 62 where annihilation radiation is detected. Accordingly, annihilation radiation is detected. PET images are generated based on detection data of annihilation radiation. In this way, PET images of a total body of the subject M are to be taken at one time.

FIG. 13 shows a timing chart of the above operations. Specifically, CT images are acquired during T1. Upon completion of imaging, the top board 52 is once returned during T2 to a state prior to taking of the CT image. PET images are then acquired during T3. Here, the top board 52 moves successively during T1 and T2. The top board 52 moves five times at five points as shown by arrows in a stepwise fashion during T3. PET images are acquired through six-time detection. No radiation is detected during movement as shown by the arrows. Here, T1 is around 1 minute, T2 is less than 1 minute, and T3 is around eighteen minutes of three minutes by six times.

[Patent Literature 1] Japanese Patent No. 3,409,506

### DISCLOSURE OF THE INVENTION

### Summary of the Invention

The conventional construction, however, has the following problem. That is, the subject M may move during an inspection, which causes difficulty in exact superimposing of both sectional images. If there is too much time between taking of a CT image and taking of a PET image, postures of the subject M in both sectional images do not conform to each other. Accordingly, positions of the subject M deviate in both sectional images. Consequently, superimposing of both sectional images cannot realize accurate mapping of radiopharmaceutical localization in the internal structure of the subject M.

This invention has been made regarding the state of the art noted above, and its object is to provide radiation tomography apparatus that allows acquisition of a sectional image having an accurately mapped radiopharmaceutical localization in an internal structure of a subject M through reduction in inspection time.

### Means for Solving the Problem

This invention discloses radiation tomography apparatus including a top board, a top board moving device, a detector ring, a PET image acquisition device, and a CT image generation device. The top board supports a subject. The top board moving device moves the top board in a top board longitudinal direction as a longitudinal direction thereof. The detector ring having a ring hole into which the top board is inserted in the top board longitudinal direction detects radiation emitted from inside of the subject. The PET image acquisition device acquires a PET image as a sectional image showing radiopharmaceutical distribution in the subject based on detection data outputted from the detector ring. The CT image generation device is provided with an introducing hole into which the top board is inserted in the top board longitudinal direction. The apparatus further includes a superimposing device for superimposing a CT image and the PET image, mentioned later. The detector ring and the CT image generation are arranged in the longitudinal direction. The CT image generation device includes a radiation source, a radiation detecting device, a rotating device, and a CT image acquisition device. The radiation source emits radiation. The radiation detecting device detects radiation emitted from the radiation source. The rotating device synchronously rotates the radiation source and the radiation detecting device about the longitudinal direction as a center axis while maintaining a relative relationship therebetween. The CT image acquisition device acquires a CT image as a sectional image showing an internal structure of the subject based on detection data outputted from the radiation detecting device. The top board moving device moves the top board in one direction along the longitudinal direction from an initial position to a termination position while stopping at given times, and simultaneously the detector ring and the radiation detecting device detect radiation at every stop of the top board. Each image acquisition device acquires each sectional image based on detection data outputted from the detector ring and the radiation detecting device when the top board is in each stop position. Each of a first width and a second width is not less than one half of a center distance. Here, let a distance between a first center and a second center be the center distance, the first center being a center of a range in the top board longitudinal direction where the detector ring acquires a PET image, and the second center being a center of a range in the top board longitudinal direction where the radiation detecting device acquires a CT image. In addition, let a width of a range in the top board longitudinal direction be the first width where the detector ring may acquire a PET image while the top board stops, and a width of a range in the top board longitudinal direction be the second width where the CT image generation device may acquire a CT image while the top board stops.

### Operation and Effect

According to this invention, the top board moves in one direction from the initial position to the termination position. Specifically, the top board stops several times between a starting position forwardly in a top board movement direction and a termination position backwardly in the top board movement direction. Both CT image and PET image are acquired during movement of the top board. When the top board lies in each stop position, the detector ring and the radiation detecting device detect radiation for outputting detection data to each image acquisition device. Each image acquisition device acquires each sectional image based on the data. In this way, according to this invention, the CT image showing the internal structure of the subject as well as the PET image showing radiopharmaceutical distribution in the subject may be generated by merely moving the top board in one direction. Accordingly, radiation tomography apparatus may be provided having reduced inspection time.

In the conventional apparatus, the CT image of the total body is acquired, and then the PET image of the total body is acquired. In contrast to this, each of the detector ring and the CT image generation device takes a sectional image in each field of view while the top board stops. The CT image and the PET image are acquired in parallel during movement of the top board in one direction, whereby both images may lead to acquisition of both sectional images of the total body of the subject. With such configuration, intervals of time for taking both sectional images may be set constant throughout the body of the subject. For instance, a CT image of the head of the subject is taken and the top board moves twice, and then a PET image is acquired for the head of the subject imaged in advance. In other words, assumed that once movement of the top board is one step, a PET image of the head is taken after two steps from taking the CT image. This relationship is similarly applicable to other portions of the subject. That is, each portion of the total body in the PET image is taken after two steps from taking the CT image corresponding to the portion.

In the conventional method, the PET image is taken six times. Accordingly, it takes eighteen minutes to finish taking all the sectional images. Sixth acquisition of a PET image starts after fifteen minutes elapse from taking all the CT images. It is difficult not to make the subject move for fifteen minutes. The subject in the PET image and that in the CT image deviate from each other in position. In contrast to this, with the configuration of this invention, a CT image taken two steps before has been acquired in every taking of the PET image of six times. Two steps correspond to around six minutes. Accordingly, the subjects falling on both sectional images do not deviate in position. Superimposing of these images may realize accurate mapping of radiopharmaceutical localization in the internal structure of the subject M.

According to the foregoing configuration, the first width as a width of the range where the detector ring may acquire a PET image and the second width as a width of the range where the CT image generation device may acquire a CT image are both set to be not less than one half of the center distance between a center of the detector ring and a center of the radiation detecting device. A field of view of the detector ring is not allowed to overlap with that of the CT image generation device due to mechanical restriction. In general, a gap is provided between both fields of view for spacing away in the top board longitudinal direction. Too large gap may prevent parallel imaging for both sectional images. On the other hand, according to this invention, each of the first width and the second width is set to be not less than the center distance. Here, the larger the width of the gap in the top board longitudinal direction becomes, the longer the center distance becomes. Taking into consideration of this, a field of view may be ensured that is sufficient to acquire both sectional images accurately even when a gap exists between both fields of view.

Moreover, each of the foregoing image acquisition devices acquires a sectional image throughout the subject by repeating acquisition of a sectional image for each section of the subject that is divided by every center distance in the top board longitudinal direction. Such configuration is more desirable.

### Operation and Effect

According to the foregoing configuration, each sectional image may be taken suitable for diagnosis. Specifically, in the foregoing configuration, the CT device and the PET device repeatedly image the same site of the subject, thereby acquiring each sectional image throughout the subject. In other words, a sectional image of the subject may be acquired for every section having a center distance in the top board longitudinal direction. Here, let the first width and the second width be not less than one half of the center distance. The CT image generation device and the detector ring each have a field of view that is certainly larger than each section of the subject. Accordingly, the foregoing configuration may realize accurate generation of the sectional image throughout the subject.

Moreover, the CT image generation device and the detector ring image the same site of the subject. Consequently, each of the CT images contains the site of the subject that is same as that taken by the PET image in the top board longitudinal direction. Accordingly, both sectional images may be superimposed with more accuracy.

The foregoing top board moving device repeats a procedure of moving the top board in one direction by a length obtained by dividing a half length of the center distance by one or more integers and then stopping the top board. Such configuration is more desirable.

Moreover, the top board moving device repeats a procedure of moving the top board in one direction by half a length of the center distance and then stopping the top board. Such configuration is more desirable.

### Operation and Effect

In general, both fields of view are not equal in width in the top board longitudinal direction. There arises a problem under such state how the top board slides for accurately acquiring both sectional images throughout the subject. One solution is to slide the top board with reference to a shorter field of view. However, this never realizes accurate superimpose of both sectional images. That is because the number of times for taking both sectional images differs from each other, which leads to deviation in the longitudinal direction in superimposing both sectional images. This invention adopts not the foregoing configuration but a configuration in which the top board moves with reference to the center distance. Such configuration may set the number of times for taking both the sectional images be equal. Consequently, each of the CT images contains the site of the subject that is same as that taken by the PET image in the top board longitudinal direction. Accordingly, both sectional images may be superimposed with more accuracy.

Moreover, it is more desirable that a selecting device is provided to selectively execute one of (α) movement of the top board, (β) radiation detection by the detector ring, and (γ) radiation detection by the radiation detecting device, exclusively.

### Operation and Effect

The foregoing configuration may realize acquisition of both sectional images with more accuracy. Both sectional images are acquired while the top board stops. Moreover, it is not desirable for the detector ring to detect an annihilation radiation pair generated from inside of the subject, since the radiation source emits radiation during radiation detection by the radiation detecting device. The foregoing configuration assures that the above three types of operations are not performed simultaneously. Consequently, a situation may be avoided where imaging cannot be conducted for each section of the subject due to movement of the top board during taking both sectional images. Moreover, a further situation may be avoided where acquisition of the PET image becomes difficult due to entering of radiation from the radiation source during acquisition of the PET image.

Moreover, further included are a period determination device for determining a period during which the subject moves as mentioned above, and a synchronizing device for correlating the determined period with imaging. Each image acquisition device acquires each sectional image only using detection data when movement of the subject lies in a phase. Such configuration is more desirable.

### Operation and Effect

According to the foregoing configuration, both sectional images may be acquired suitable for diagnosis. Each sectional image is taken in synchronization with movement of the subject. Such configuration may realize acquisition of both sectional images independently of movement of the subject.

### Effect of the Invention

According to this invention, the top board stops several times between a starting position forwardly in a top board movement direction and a termination position backwardly in the top board movement direction. Both CT image and the PET image are acquired during movement of the top board. When the top board lies in each stop position, the detector ring and the radiation detecting device detect radiation for outputting detection data to each image acquisition device. Each image acquisition device acquires each sectional image based on the data. In this way, according to this invention, the CT image showing the internal structure of the subject as well as the PET image showing radiopharmaceutical distribution in the subject may be generated by merely moving the top board in one direction.

In the conventional method, the PET image is taken six times. Accordingly, it takes eighteen minutes to finish taking all the sectional images. Sixth acquisition of a PET image starts after fifteen minutes elapse from taking all the CT images. It is difficult not to make the subject move for fifteen minutes. The subject in the PET image and that in the CT image deviate from each other in position. In contrast to this, with the configuration of this invention, assumed that once movement of the top board is one step, a CT image taken two steps before has been acquired in every taking of the PET image of six times. Two steps correspond to around six minutes. Accordingly, the subjects falling on both sectional images do not deviate in position. Superimposing of these images may realize accurate mapping of radiopharmaceutical localization in the internal structure of the subject M.

### BRIEF DESCRIPTION OF THE DRAWINGS

FIGS. 1 and 2 are functional block diagrams each showing a configuration of radiation tomography apparatus according to Embodiment 1.

FIG. 3 is a perspective view showing a configuration of a radiation detector according to Embodiment 1.

FIG. 4 is a functional block diagram showing a configuration of a collimator according to Embodiment 1.

FIGS. 5 and 6 are schematic views each showing a relationship between a field of view and a center distance according to Embodiment 1.

FIG. 7 is a sectional view showing operations of the radiation tomography apparatus according to Embodiment 1.

FIG. 8 is a timing chart showing operations of the radiation tomography apparatus according to Embodiment 1.

FIGS. 9 and 10 are schematic views each showing operations of the radiation tomography apparatus according to Embodiment 1.

FIG. 11 is a functional block diagram showing radiation tomography apparatus according to one modification.

FIG. 12 is a sectional view showing a configuration of conventional radiation tomography apparatus.

FIG. 13 is a timing chart showing the configuration of the conventional radiation tomography apparatus.

### DESCRIPTION OF REFERENCES

C . . . center distance
Fa . . . first width
Fb . . . second width
3 . . . X-ray tube, radiation source
4 . . . FPD (radiation detecting device)
8 . . . CT device (image generation device)
10 . . . top board
12 . . . detector ring
15 . . . top board moving mechanism (top board moving device)
24 . . . PET image acquisition section (PET image acquisition device)
25 . . . CT image acquisition section (CT image acquisition device)
26 . . . superimposing section (superimposing device)
31 . . . rotating mechanism (rotating device)
38 . . . selecting section (selecting device)
46 . . . period determining section (period determining device)
47 . . . synchronizing section (synchronizing device)

### Embodiment 1

Description will be given hereinafter of radiation tomography apparatus 1 according to Embodiment 1 with reference to the drawings.

<Configuration of Radiation Tomography Apparatus>

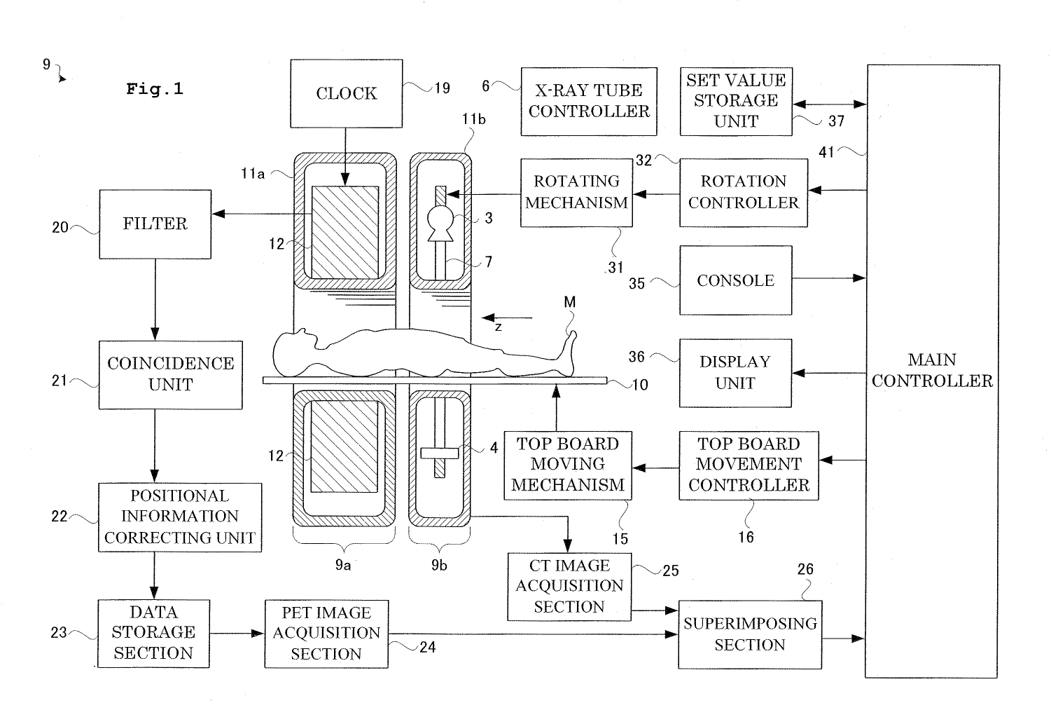

Each embodiment of radiation tomography apparatus according to this invention will be described hereinafter with reference to the drawings. FIG. 1 is a functional block diagram showing a configuration of radiation tomography apparatus according to Embodiment 1. As shown in FIG. 1, the radiation tomography apparatus 9 according to Embodiment 1 includes a top board 10 for placing a subject M on the back thereof. Radiation tomography apparatus 9 further includes a PET device 9a for imaging radiopharmaceutical distribution in the subject and a CT device 9b for imaging an internal structure, such as internal organs, of the subject. The PET device 9a and the CT device 9b are arranged in parallel in a z-direction (i.e., a top board longitudinal direction as a longitudinal direction of the top board 10, a direction of a body axis of the subject M.) The PET device 9a and the CT device 9b each include an introducing hole into which the top board 10 is inserted in the z-direction. Each introducing hole has a cylindrical shape extending in the z-direction. The CT device 9a corresponds to the CT image generation device in this invention.

The PET device 9a and the CT device 9b have a gantry 11a and 11b, respectively, with a through hole for surrounding the subject M. The top board 10 is provided as to pass through an opening of the gantry 11a and 11b. The top board 10 freely moves in and out along the z-direction. A top board moving mechanism 15 slides the top board 10 as above. A top board movement controller 16 controls the top board moving mechanism 15. The top board moving mechanism 15 corresponds to the top board moving device in this invention. The top board movement controller 16 corresponds to the top board movement control device for controlling the top board moving mechanism 15.

The PET device 9a includes a detector ring 12 inside thereof that detects annihilation gamma-ray pairs from the subject M. The detector ring 12 is tubular extending in the z-direction. The detector ring 12 has a length of around 30 cm in the z-direction. A clock 19 sends temporal information with a serial number to the detector ring 12 and a synchronizing section 47, mentioned later. Temporal information about when gamma rays were acquired is added to detection data outputted from the detector ring 12, and then inputted into a filter 20, mentioned later.

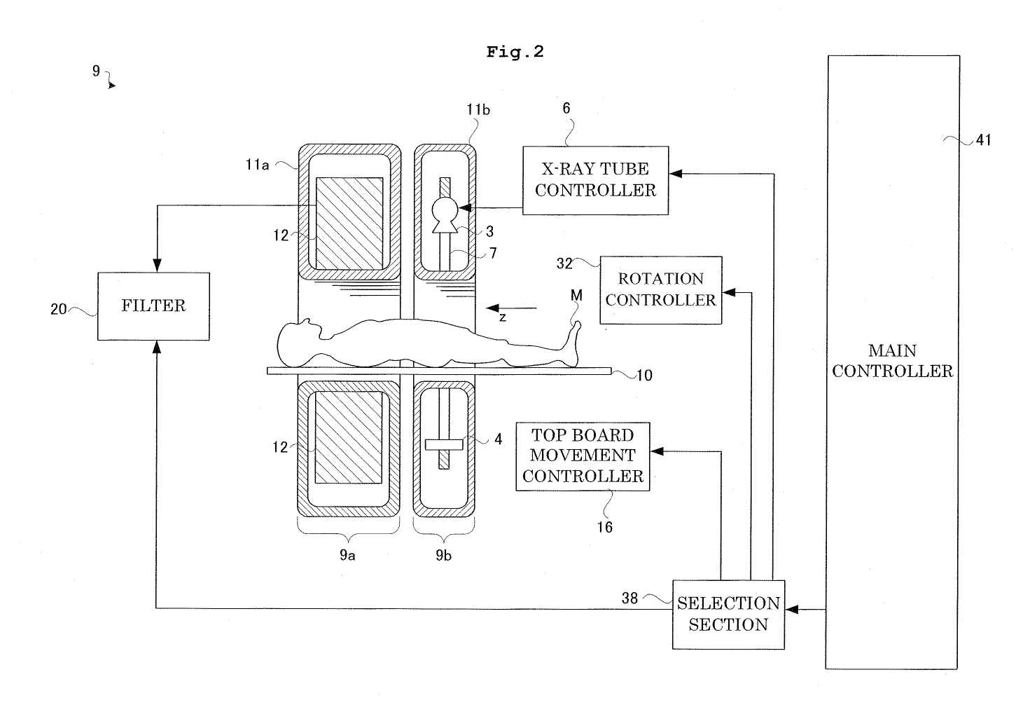

The selecting section 38 shown in FIG. 2 is provided as to make the X-ray tube controller 6, the top board movement controller 16, the filter 20, and a rotation controller 32 operate sequentially. Specifically, the selecting section 38 (1) does not make each section 6, 20, 32 operate while the top board movement controller 16 slides the top board 10 in the z-direction, (2) does not make each section 6, 16, 32 operate while the filter 20 obtains detection data from the detector ring 12, and (3) does not make each section 16, 20 operate while the X-ray tube controller 6 and the rotation controller 32 cooperate to acquire a CT image of the subject. In so doing, the selecting section 38 does not perform slide of the top board, CT imaging, and PET imaging simultaneously. That is, the selecting section 38 selectively executes one of (a) slide of the top board 10, (β) annihilation radiation detection by the detector ring 12 (PET imaging), and (γ) radiation detection by the FPD 4 (CT imaging), exclusively. Here, no operation of the filter 20 means that the filter 20 does not pass detection data from the detector ring 12 to a subsequent coincidence unit 21. The FPD 4 corresponds to the radiation detecting device in this invention. The selecting section 38 corresponds to the selecting device in this invention.

Description will be given of a configuration of the detector ring 12. According to Embodiment 1, around one hundred radiation detectors 1 are arranged along an imaginary circle on a plane perpendicular to the z-direction, whereby one unit ring is formed. The unit rings are arranged in the z-direction to form the detector ring 12.

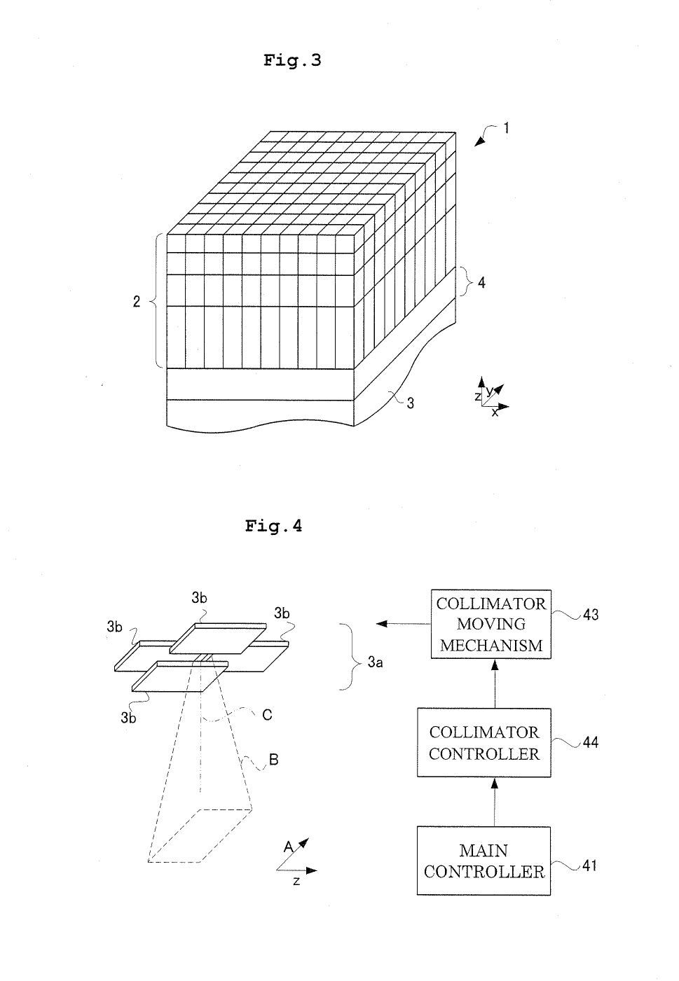

Next, simple description will be given of a configuration of the radiation detector 1. FIG. 3 is a perspective view showing a configuration of the radiation detector according to Embodiment 1. As shown in FIG. 3, the radiation detector 1 includes a scintillator 2 for converting radiation into fluorescence, and a light detector 3 for detecting fluorescence. A light guide 4 is provided between the scintillator 2 and the light detector 3 for receiving fluorescence.

The scintillator 2 has scintillation counter crystals arranged in a two-dimensional array. Each of the scintillation counter crystals C is composed of Ce-doped Lu_{2(1-X)}Y_{2X}SiOₛ (hereinafter referred to as LYSO.) The light detector 3 allows determination about which scintillation counter crystal emits fluorescence as well as intensity of fluorescence and time when fluorescence is generated. Here, the scintillator 2 having the configuration of Embodiment 1 is only exemplification of an aspect that may be adopted. Consequently, the configuration of this invention is not limited to this.

Detection data outputted from the detector ring 12 is sent to the coincidence unit 21 (see FIG. 1) via the filter section 20. Two gamma rays entering into the detector ring 12 simultaneously correspond to an annihilation radiation pair through radiopharmaceutical in the subject. The coincidence unit 21 counts the number of detecting an annihilation radiation pair for every combination of two scintillation counter crystals that form the detector ring 12. The result is memorized into to a positional information correcting unit 22. A positional relationship of the scintillation counter crystals upon coincidence shows incidence position and direction where the annihilation radiation pair entered into the detector ring 12, and is information necessary for mapping radiopharmaceutical. The number of detecting an annihilation radiation pair and energy intensity in the annihilation radiation that is memorized for every combination of the scintillation counter crystals shows variation in occurrence of an annihilation radiation pair in the subject. This is information necessary for mapping radiopharmaceutical. Here, temporal information that the clock 19 adds to the detection data is used for determination of coincident property of detection data by the coincidence unit 21.

The top board 10 moves in the z-direction relative to the detector ring 12. Accordingly, a positional relationship deviates between the subject M and the detector ring 12. The positional information correcting unit 22 corrects the deviation. The top board movement control section 16 sends to the positional information correcting unit 22 signals showing a moving state of the top board 10. The positional information correcting unit 22 corrects positional information components in coincidence data sent from the coincidence unit 21 in accordance with the signals. Specifically, the positional information correcting unit 22 shifts positional information components in coincidence data as to follow movement of the top board 10 in the z-direction. The corrected coincidence data is memorized into a data storage section 23.

The coincidence counting data is sent to a PET image acquisition section 24. Then, coincidence data is mapped in three dimensions. Consequently, two or more axial images (sliced images in a plane perpendicular to the z-direction) of the subject M may be acquired. This process is referred to as PET imaging in this invention. The sectional image acquired by the PET image acquisition section 24 shows radiopharmaceutical distribution in the subject, and is appropriately referred to as a PET image. The PET image acquisition section 24 corresponds to the PET image acquisition device in this invention.

Next, description will be given of a configuration of a CT device 9b (see FIG. 1.) The gantry 11b of the CT device 9b has an X-ray tube 3 inside thereof for irradiating the subject M with X-rays, an FPD (flat panel detector) 4, and a support portion 7 for supporting the X-ray tube 3 and the FPD 4. The support portion 7 has a ring shape, and freely rotates about an axis parallel to the z-direction. A rotating mechanism 31 formed of a power generation device such as a motor and a power transmission device such as a gear performs rotation of the support portion 7. A rotation controller 32 controls the rotating mechanism 31. The X-ray tube controller 6 controls the X-ray tube 3. The rotating mechanism 31 corresponds to the rotating device in this invention.

The X-ray tube 3 and the FPD 4 rotate about the axis parallel to the z-direction. The X-ray tube 3 intermittently irradiates the subject M with X-rays in accordance with control of the X-ray tube controller 6. The FPD 4 detects X-rays transmitting through the subject M at every X-ray irradiation. Detection data outputted from the FPD 4 is sent to a CT image acquisition section 25. The CT image acquisition section 25 acquires a fluoroscopic image, at every X-ray irradiation, on which a fluoroscopic image of the subject M falls. An image of subject falls on a series of fluoroscopic images as acquired above while a direction of imaging the subject varies. The CT image acquisition section 25 performs reconstruction for a series of fluoroscopic images through a method such as a back projection method. Consequently, two or more axial images (sliced images in a plane perpendicular to the z-direction) of the subject M may be acquired. This process is referred to as CT imaging in this invention. An axial image acquired here shows an extent of attenuating applied X-rays during transmitting through the subject. The axial image has internal organs or a bone shape of the subject M falling thereon. Such axial image is appropriately referred to as a CT image in distinction from the above PET image. The CT image acquisition section 25 corresponds to the CT image acquisition device in this invention.

As shown in FIG. 4, the X-ray tube 3 has a collimator 3a. The collimator 3a is attached to the X-ray tube 3, and collimates X-rays from the X-ray tube 3 to generate X-ray beams B in a quadrangular pyramid shape. Description will be given in detail of the collimator 3a. As shown in FIG. 4, the collimator 3a has one pair of leaves 3b that moves in a mirror-image symmetrical manner, and has one more pair of leaves 3b that similarly moves in a mirror-image symmetrical manner. The collimator 3a moves the leaves 3b which realizes not only irradiation of an entire X-ray detection surface of the FPD 4 with X-ray beams B in a cone shape, and but also irradiation of only a center portion of the FPD 4 with X-ray beams B in a fan shape. Here, the X-ray beams B have a central axis C set therein that extends from the X-ray tube 3 toward the FPD 4. Each leaf 3b moves with reference to the central axis C in a mirror-image symmetrical manner. One pair of leaves 3b adjusts spread of X-ray beams in a quadrangular pyramid shape in the body axis direction A (z-direction), and the other pair of leaves 3b adjusts spread of X-ray beams in a direction perpendicular to the center axis C and the z-direction. A collimator moving mechanism 43 changes opening of the collimator 3a. A collimator controller 44 controls the collimator moving mechanism 43.

The radiation tomography apparatus 9 in Embodiment 1 acquires a CT image for each PET image having a same cutting position as the PET image in the z-direction. A superimposing section 26 (see FIG. 1) superimposes the CT image on the PET image each having the same cutting position in the z-direction. Accordingly, a superimposed sectional image may be acquired having mapped radiopharmaceutical distribution in the internal structure of the subject. The superimposing section 26 corresponds to the superimposing device in this invention.

The radiation tomography apparatus 9 further includes a main controller 41 for controlling each section en bloc, and a display unit 36 for displaying a radiological image. The main controller 41 has a CPU, and realizes each section 6, 16, 20, 21, 22, 23, 24, 25, 26, 31, 44 by executing various programs. The above sections may each be divided into a controller that performs their functions.

A set value storage unit 37 memorizes various parameters with respect to a movement speed of the top board 10, and control of the X-ray tube 3 and the support portion 7. A console 35 is provided for inputting operator's various instructions.

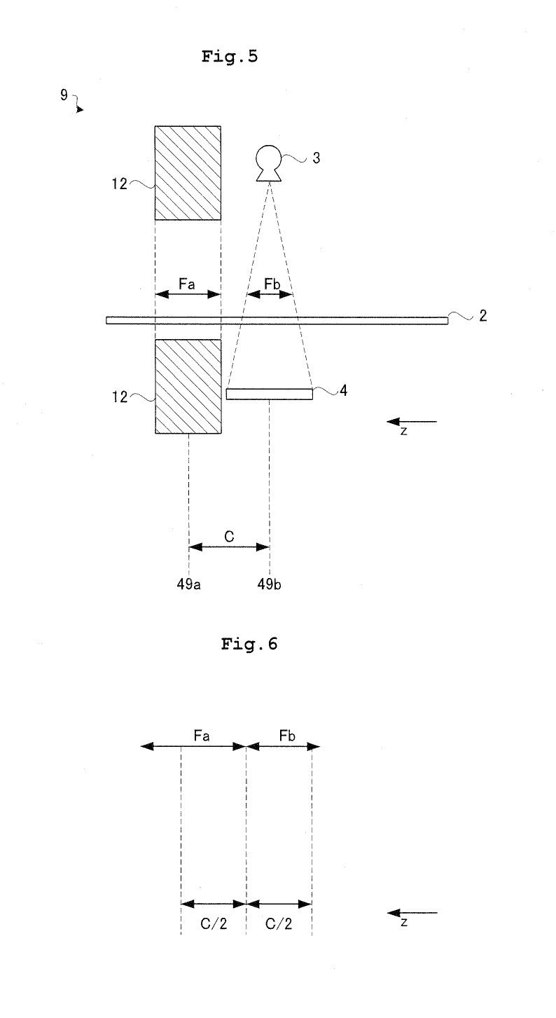

Here, description will be given of each field of view of the PET device 9a and the CT device 9b. As shown in FIG. 5, the detector ring 12 of the PET device 9a has a wide field of view in the z-direction (see Fa.) The PET device 9a acquires radiation detection data on a portion of the subject M located in the field of view. Two or more PET images are acquired such that the portion of the subject M is sliced into several sheets. On the other hand, as shown in FIG. 5, the CT device 9b has a wide field of view in the z-direction (see Fb.) The CT device 9b acquires radiation detection data on a portion of the subject M located in the field of view. Two or more CT images are acquired such that the portion of the subject is sliced into several sheets at the same position as the PET image in z-direction. Here, a length of the field of view of the detector ring 12 in the z-direction is a first width Fa in this invention. A length of the field of view of the CT device 9b in the z-direction is a second width Fb in this invention. Moreover, a center of the field of view of the detector ring 12 in the z-direction is referred to as a first center 49a. A center of the field of view of the CT device 9b in the z-direction is referred to as a second center 49b. It may be considered that movement of the top board 10 realizes a wider field of view of the PET device 9a and the CT device 9b. However, the foregoing field of view is obtained under no movement of the top board 10. Accordingly, hereinafter, the field of view of the PET device 9a (detector ring 12) refers to a region having a first width Fa as shown in FIG. 5. Similarly, the field of view of the CT device 9b refers to a region having a second width Fb as shown in FIG. 5.

A distance in the z-direction from the first center 49a to the second center 49b is a center distance C. There is a relationship as under among the center distance C, the first width Fa, and the second width Fb. As shown in FIG. 6, each of the first width Fa and the second width Fb is set to be not less than half of the center distance C. The meaning for such setting is to be mentioned later.

<Operation of Radiation Tomography Apparatus>

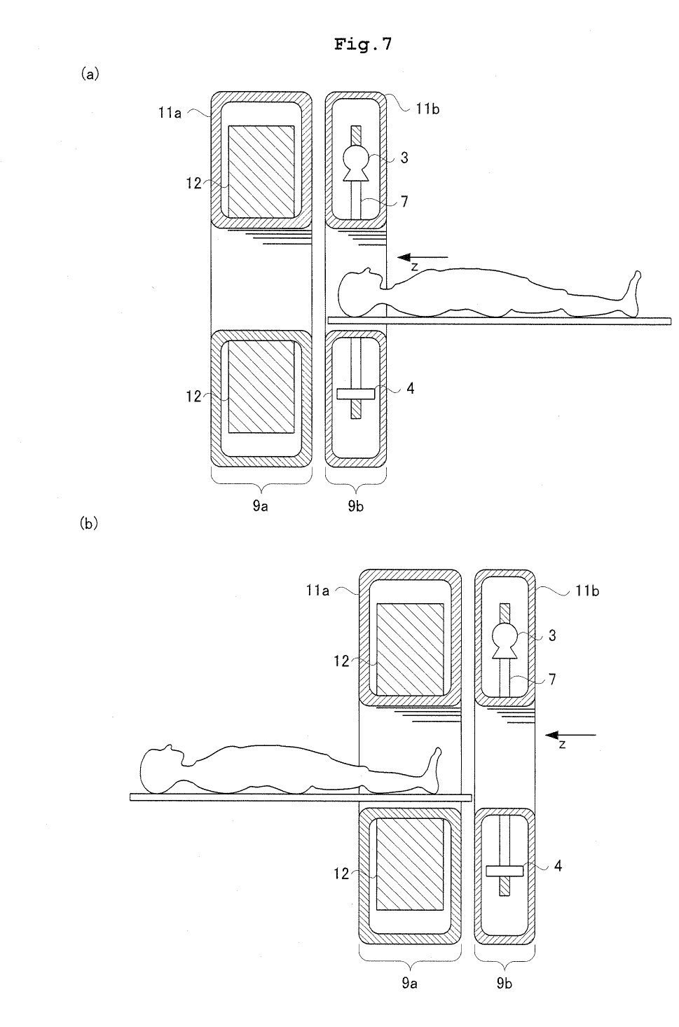

Next, description will be given of operations of the radiation tomography apparatus 9. In the radiation tomography apparatus 9, radiopharmaceutical is firstly administered by injection to the subject M for determination of radiopharmaceutical distribution in the subject M. The subject M is placed on the top board 10 after a lapse of given time from this. An operator instructs the radiation tomography apparatus 9 via the console 35 to start inspection. The top movement controller 16 controls the top board 10 as to slide in the z-direction with the subject M placed thereon. Then, the subject M is slid into a position as shown in FIG. 7(a). This position of the subject M is referred to as an initial position. Specifically, the head of the subject M entirely lies in the field of view of the CT device 9b. Thereafter, the top board 10 repeatedly slides and stops to move the subject M into a position shown in FIG. 7(b). This position of the subject M is referred to as a termination position. Specifically, the tip of the foot of the subject M lies in the filed of view of the detector ring 12.

Description will be given of movement of the top board 10 during operation. The top board 10 moves seven times from the initial position (bed position 1) in FIG. 7(a) into the termination position (bed position 8) in FIG. 7(b). That is, the top board 10 repeatedly moves and stops alternately until it reaches the termination position. In addition, the top board 10 in the initial position moves only in one direction from the initial position toward the termination position. In other words, the top board 10 moves in the one direction alone the z-direction. Accordingly, the top board 10 never moves in reverse direction. Moreover, the top board 10 slides in the z-direction by a width C/2 at one time. This is to be repeated seven times.

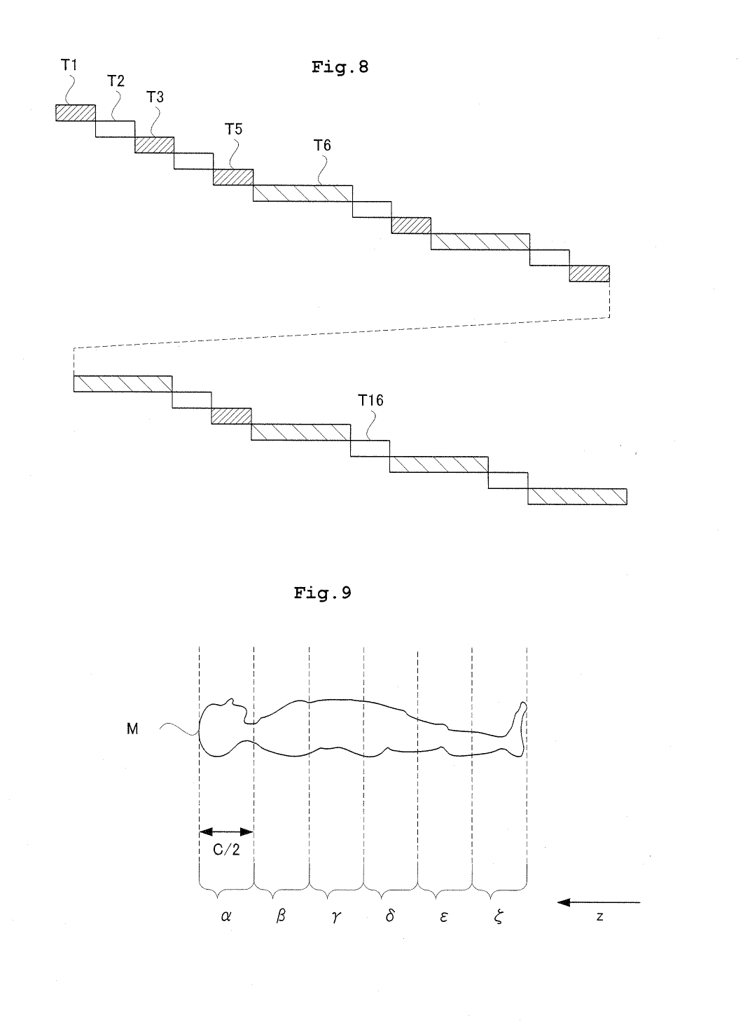

FIG. 8 is a timing chart showing operations of the radiation tomography apparatus 9 according to Embodiment 1. The fine right-diagonally shaded areas in the drawing indicates a period of time for CT imaging, whereas the coarse left-diagonally areas in the drawing indicates a period of time for PET imaging. The open area having no diagonal indicates a period of time for sliding the top board 10. It takes not more than one second to conduct CT imaging at one time, whereas it takes around three minutes to conduct PET imaging at one time. When the top board 10 slides to the initial position, the top board movement controller 16 sends to the selection section 38 information that slide has been completed. The selection section 38 executes once rotation of the X-ray tube 3 and the FPD4 while the top board 10 stops. In this way, a CT image of the head of the subject M is acquired (T1 in FIG. 8.) Hereafter, CT imaging is conducted to the subject six times. Specifically, CT imaging is conducted for six sections obtained through dividing the subject M into six portions by a width C/2 in the z-direction (see FIG. 9.) Each section is referred to as a subject section.

After first CT imaging completes, the selection section 38 starts operation of the top board movement controller 16. Thus, the top board 10 slides forwardly in the z-direction by C/2 (T2 in FIG. 8.) Thereafter, the same operation as T1 and T2 is repeated once again. After completion of this, CT imaging is conducted once again (T5 in FIG. 8.) In so doing, CT imaging is completed for a first section a to a third section γ of the six subject sections (see FIG. 9.)

Subsequently, the detector ring 12 detects an annihilation radiation pair in the head of the subject M without sliding the top board 10. Thus, a PET image is acquired for the head of the subject M (T6 in FIG. 8.) Hereinafter, this operation is referred to as PET imagine. Thereafter, sliding of the top board 10, CT imaging, and PET imaging is repeated, in this order, three times. At this time, CT imaging is completed for every section of the six subject sections, and PET imaging is completed for the first section a to the third section y (see FIG. 9.)

Subsequently, the top board 10 slides by C/2 in the z-direction (T16 in FIG. 8), and then PET imaging is conducted. Thereafter, sliding of the top board and PET imaging is repeated once again. In so doing, radiation detection with the radiation tomography apparatus 9 according to Embodiment 1 is to be completed. In other words, PET imaging is to be completed at this time for every section of the six subject sections.

The superimposing section 26 superimposes the acquired CT images and the PET images to acquire a superimposed sectional image. This superimposed sectional image is displayed on the display unit 36 to complete an inspection.

Now, description will be given of the meaning of setting a length of the first width Fa and the second width Fb. For simple explanation, it is assumed that the subject M is divided into six subject sections α to ζ divided in the z-direction by a width C/2 as shown in FIG. 9. The subject sections α to ζ are sections obtained by dividing the subject by a width of the center distance along the z-direction.

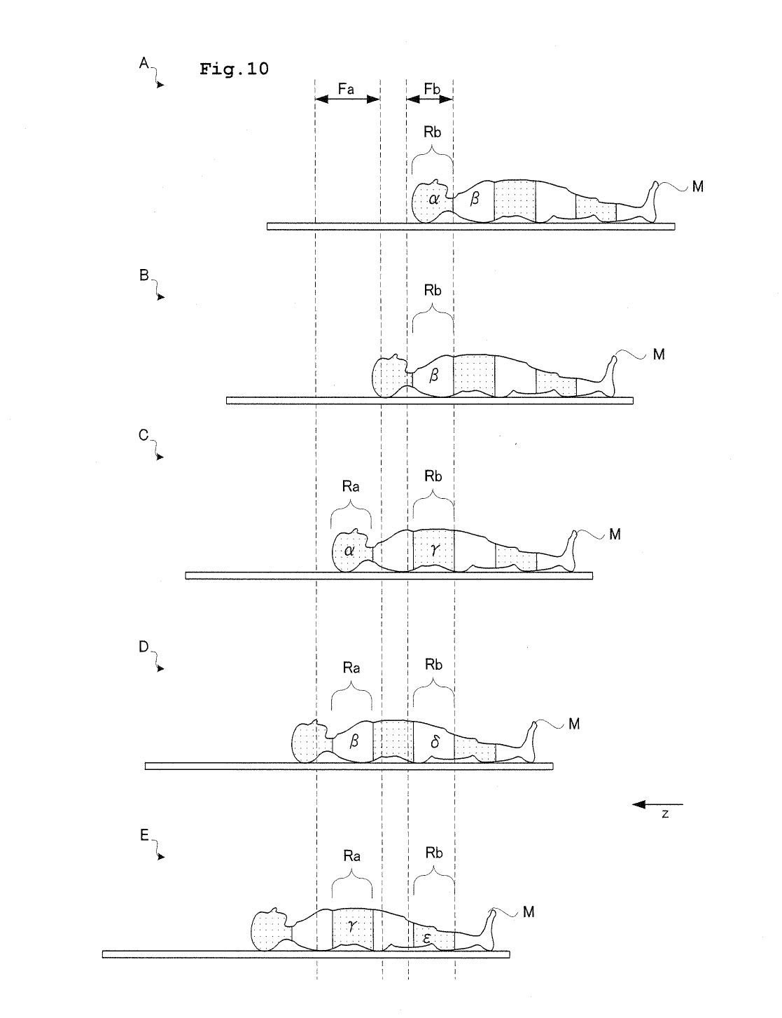

FIG. 10 shows introduction of the subject N4 into each field of view. A symbol “A” in FIG. 10 shows a relationship between the subject M and each field of view at a timing T1 in FIG. 8. The subject section a has a width C/2 in the z-direction. Accordingly, the entire subject section a certainly lies within the field of view of the CT device 9b having a large width than C/2 in the z-direction. At a timing T1, let a wide section in the z-direction be Rb where the subject section a lies.

Subsequently, the top board 10 slides by each C/2. The subject M is moved by C/2 in the z-direction at every slide thereof. The subject sections α to ζ each have the same width as a moving distance of C/2. The subject sections β, γ, δ, ε are located within the section Rb in turn in this order at every slide of the top board 10. This situation is illustrated as “A” to “E” in FIG. 10. That is, the top board 10 moves five times by C/2 in the z-direction from a state “A” in FIG. 10, whereby the subject sections α to κε are located within the section Rb in turn. Here, the top board 10 stops while the subject sections α to ζ are located within the section Rb. Specifically, the CT device 9a sequentially takes a CT image for every section having a width C/2 when the subject sections α to ζ are located within the section Rb.

In a state C in FIG. 10, the top board has slid twice. Here, the subject section a lies within the filed of view of the detector ring 12. Such slide of the top board 10 may lead to a state where the entire subject section α lies within the filed of view in the detector ring 12 and the top board 10 stops. Here, the top board 10 moves by each C/2 in the z-direction. Moreover, the subject sections α to ζ each have the same width as a moving distance of C/2. Accordingly, the entire subject section a certainly lies within the filed of view of the detector ring having a larger width than C/2 in the z-direction. Actually, the entire subject section a lies within the field of view of the detector ring 12 when the top board 10 has slid twice (i.e., the top board 10 stops in a bed position 3.) At this time, let a wide section in the z-direction be Ra where the subject section α lies. See C in FIG. 10.

Subsequently, the top board 10 slides by each C/2. The subject M is moved by C/2 in the z-direction at every slide thereof. The subject sections each have the same width as a moving distance of C/2. The subject sections β, γ are located within the section Ra in turn in this order in every slide of the top board 10. This situation is illustrated as C to E in FIG. 7. That is, the top board 10 moves five times by C/2 in the z-direction from a state C in FIG. 10, whereby the subject sections α to ζ are located within the section Ra in turn. Here, the top board 10 stops while the subject sections α to ζ are located within the section Ra. Specifically, the radiation tomography apparatus 9 sequentially takes a PET image for every section having a width C/2 when the subject sections α to ζ are located within the section Ra.

As above, the first width Fa and the second width Fb each have a length not less than C/2. Moreover, the top board 10 slides by C/2. Consequently, the radiation tomography apparatus 9 may acquire a CT image and a PET image for every subject section α to ζ. Such configuration may realize CT imaging for every subject section α to ζ under a controlled condition. Moreover, PET imaging may be conducted for every subject section α to ζ under a controlled condition. Consequently, a superimposed image may be acquired suitable for diagnosis.

Here, the first width Fa and the second width Fb have a length not less than C/2. Accordingly, when each of the subject sections α to ζ is imaged, a sectional image contains the entire section to be imaged and a portion of the section adjacent thereto that are simultaneously imaged. The subject sections α to ζ overlap one another in the z-direction to take both sectional images. Consequently, it is ensured that the subject M is entirely imaged with no discontinuity of the images within each subject section α to ζ. Moreover, X-ray beams may be restricted as to have a minimum width required for acquisition of a CT image in the section Rb for the purpose of suppressing radiation exposure to the subject. In this case, the field of view Fb as a width in the z-direction equal to C/2. Such consideration is unnecessary with respect to the first width Fa.

According to Embodiment 1, the top board 10 moves in one direction from the initial position to the termination position. Specifically, the top board 10 stops several times between a starting position forwardly in a top board movement direction and a termination position backwardly in the top board movement direction. Both CT image and the PET image are acquired during movement of the top board 10. When the top board 10 lies in each stop position, the detector ring 12 and the FPD 4 detect radiation for outputting detection data to each image acquisition section 24, 25. Each image acquisition section 24, 25 acquires each sectional image based on the data. In this way, according to Embodiment 1, the CT image showing the internal structure of the subject as well as the PET image showing radiopharmaceutical distribution in the subject may be generated by merely moving the top board 10 in one direction. Accordingly, radiation tomography apparatus may be provided having reduced inspection time.

In the conventional apparatus, the CT image of the total body is acquired, and then the PET image of the total body is acquired. In contrast to this, in Embodiment 1, each of the detector ring 12 and the CT device 9b take a sectional image in each bed position. That is, the CT image and the PET image are acquired in parallel during movement of the top board 10 in one direction, which may lead to acquisition of both sectional images of the total body of the subject. With such configuration, intervals of time for taking both sectional images may be set constant throughout the body of the subject. Specifically, a CT image is taken for the subject section α and the top board 10 moves twice, and then a PET image is taken for the subject section α. In other words, assumed that once movement of the top board 10 is one step, a PET image for the subject section α is conducted after two steps from taking the CT image. This relationship is similarly applicable to other subject sections β to ζ. That is, each portion of the total body in the PET image is taken after two steps from taking the CT image corresponding to each portion.

In the conventional method, the PET image is taken six times. Accordingly, it takes eighteen minutes to finish taking all the sectional images. Sixth acquisition of a PET image starts after fifteen minutes elapse from taking all the CT images. It is difficult not to make the subject move for fifteen minutes. The subject in the PET image and that in the CT image deviate from each other in position. In contrast to this, with the configuration of Embodiment 1, a CT image taken two steps before has been acquired in every taking of the PET image of six times. Two steps correspond to around six minutes. Accordingly, the subjects falling on both sectional images do not deviate in position. Superimposing of these images may realize accurate mapping of radiopharmaceutical localization in the internal structure of the subject M.

According to Embodiment 1, the first width Fa as a width of the range where the detector ring 12 may acquire a PET image and the second width as a width Fb of the range where the CT device 9b may acquire a CT image are both set to be not less than one half of the center distance C between a center of the detector ring 12 and a center of the FPD 4. A field of view of the detector ring 12 is not allowed to overlap with that of the CT device 9b in the z-direction due to mechanical restriction. In general, a gap is provided between both fields of view for spacing away in the z-direction. A too large gap may prevent parallel imaging for both sectional images. On the other hand, according to Embodiment 1, each of the first width Fa and the second width Fb is set to be not less than the center distance. Here, the larger the width of the gap in the z-direction becomes, the longer the center distance C becomes. Taking into consideration of this, a field of view may be ensured that is sufficient to acquire both sectional images accurately even when a gap exists between both fields of view.

According to Embodiment 1, each sectional image may be taken suitable for diagnosis. Specifically, in Embodiment 1, the CT device 9b and the detector ring 12 repeatedly image the same site of the subject, thereby acquiring each sectional image throughout the subject. In other words, in Embodiment 1, a sectional image of the subject may be acquired for every section having a center distance C. Here, let the first width Fa and the second width Fb be not less than one half of the center distance C. The CT device 9b and the detector ring 12 each have a field of view that is certainly larger than each section of the subject. Accordingly, Embodiment 1 may realize accurate generation of the sectional image throughout the subject.

Moreover, the CT device 9b and the detector ring 12 image the same site of the subject. Consequently, each of the CT images contains the site of the subject that is same as that taken by the PET image in the z-direction. Accordingly, both sectional images may be superimposed with more accuracy.

In general, both fields of view are not equal in width in the z-direction. There arises a problem under such state how the top board 10 slides for accurately acquiring both sectional images throughout the subject M. One solution is to slide the top board 10 with reference to a shorter field of view. However, this never realizes accurate superimpose of both sectional images. That is because the number of times for taking both sectional images differs from each other, which leads to deviation in the z-direction in superimposing both sectional images. In Embodiment 1, the top board 10 moves with reference to the center distance C. Such configuration may set the number of times for taking both the sectional images be equal. Consequently, each of the CT images contains each subject section a to C same as that taken by the PET image in the z-direction. Accordingly, both sectional images may be superimposed with more accuracy.

Embodiment 1 may realize acquisition of both sectional images with more accuracy. Both sectional images are acquired while the top board 10 stops. Moreover, it is not desirable for the detector ring 12 to detect an annihilation radiation pair generated from inside of the subject, since the radiation source emits radiation during radiation detection by the FPD 4. Embodiment 1 assures that the above three types of operations are not performed simultaneously. Consequently, a situation may be avoided where imaging cannot be conducted for each section of the subject due to movement of the top board 10 during taking both sectional images. Moreover, a further situation may be avoided where acquisition of the PET image becomes difficult due to entering of radiation from the X-ray tube 3 during acquisition of the PET image.

This invention is not limited to the foregoing configurations, but may be modified as follows.

(1) Embodiment 1 starts with imaging by the CT device 9b. This invention is not limited to this configuration. Embodiment 1 may start with taking of a PET image through changing a sliding direction of the top board 10 or a placement direction of the subject M.

(2) In Embodiment 1, the top board 10 slides by C/2 in the z-direction at one time. Alternatively, the top board 10 may slide by C/2 several times. That is, the top board 10 slides by C/2n in the z-direction, and then stops. Repeating slide and stop as above may realize acquisition of the sectional image for the total body. Here, it is desirable to limit n to one or more integers. In so doing, the subject M is divided in the z-direction per unit C/2 for acquisition of each sectional image.

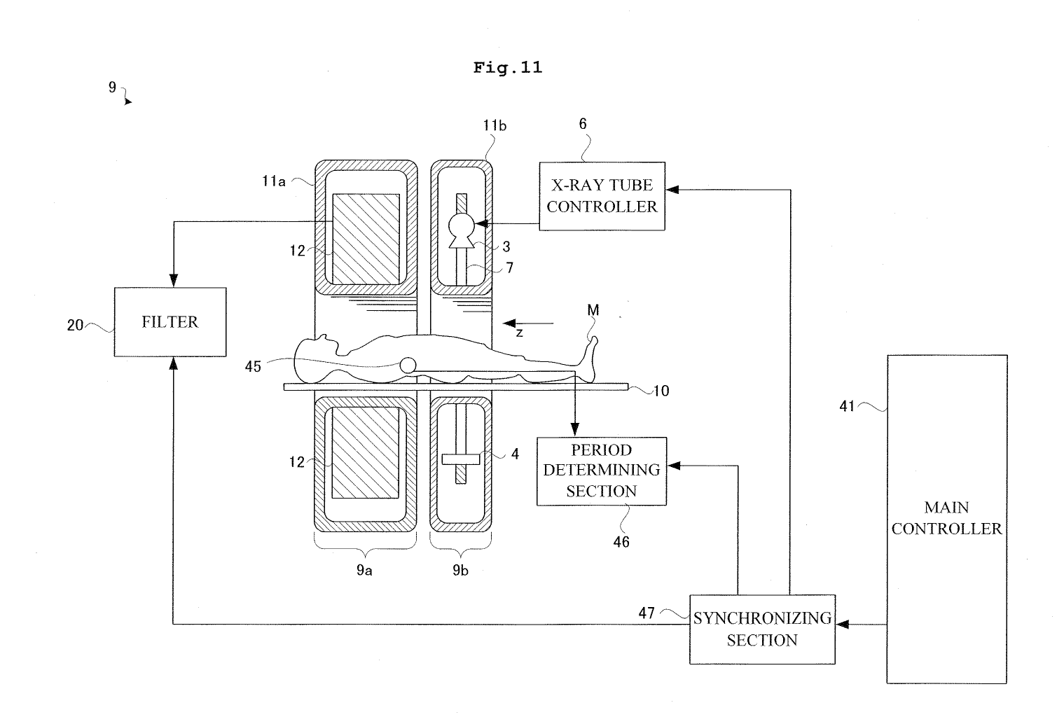

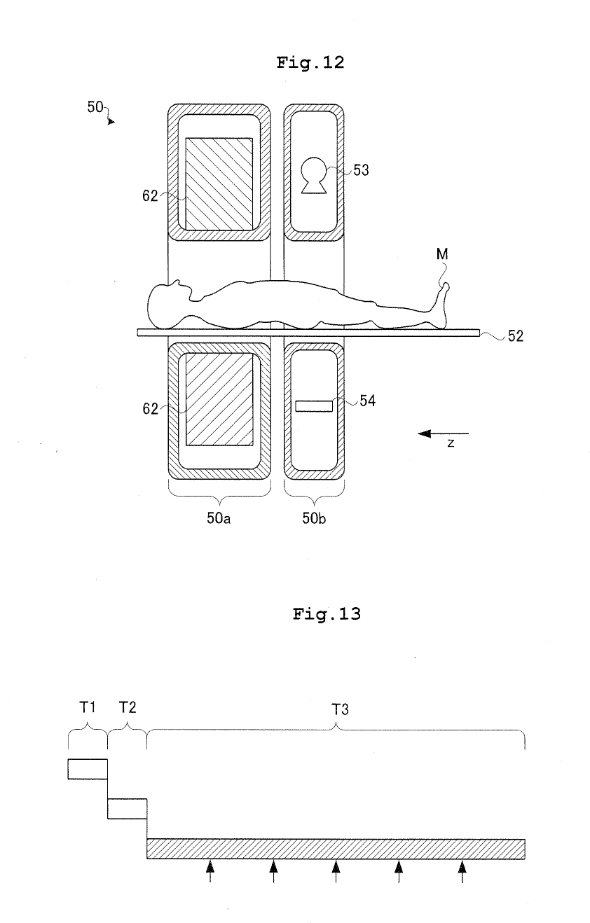

(3) In addition to the configuration of Embodiment 1, imaging may be conducted taking into consideration of periodic movement of the subject. As shown in FIG. 11, this modification includes a sensor 45 for sensing movement of the subject M, a period determining section 46 for determining a period of movement in accordance with sensor signals outputted from the sensor 45, and a synchronizing section 47 for correlating period data outputted from the period determining section 46 with detection data on imaging. Here, movement of the subject may occur from such as breathing and cardiac beats. The synchronizing section 47 corresponds to the synchronizing device in this invention. The period determining section 46 corresponds to the period determining device in this invention.

The synchronizing section 47 enables and disenables irradiation with X-rays from the X-ray tube controller 6 periodically. In so doing, a series of fluoroscopic images may be acquired, for example, having an image falling thereon when the subject inhales air to the maximum extent. The CT image acquisition section 25 (see FIG. 1) acquires a CT image having periodicity of movement being correlated therewith in accordance with the fluoroscopic images. Moreover, the synchronizing section 47 sends periodic data to the filter section 20 (see FIG. 1.) The filter 20 adds periodic data to detection data outputted from the detector ring 12. The PET image acquisition section 24 (see FIG. 1) acquires a PET image using only detection data observed when the subject inhales air to the maximum extent. Superimposing both sectional images as acquired above may realize acquisition of a superimposed sectional image under consideration of movement of the subject. In so doing, the superimposed sectional image becomes clearer. In the above explanation, a phase of acquiring a sectional image is time when the subject inhales air to the maximum extent. An operator may select via a console 35 a phase of acquiring a sectional image. According to this modification, both sectional images may be acquired suitable for diagnosis. Each sectional image is taken in synchronization with movement of the subject. Such configuration may realize acquisition of both sectional images independently of movement of the subject.

(4) In each of the foregoing embodiments, the scintillation counter crystal is composed of LYSO. Alternatively, the scintillation counter crystal may be composed of another materials, such as GSO (Gd₂SiO₅), in this invention. According to this modification, a method of manufacturing a radiation detector may be provide that allows provision of a radiation detector of low price.

(5) The fluorescence detector in each of the foregoing embodiments is formed of the photomultiplier tube. A photodiode, an avalanche photodiode, a semiconductor detector, etc., may be used instead of the photomultiplier tube.

(6) In the foregoing embodiments, the top board 10 slides five times. Alternatively, frequency of slide may vary in accordance with setting of the center distance C.

### INDUSTRIAL UTILITY

As described above, this invention is suitable for radiation tomography apparatus for medical uses.

1. Radiation tomography apparatus comprising:
a top board for supporting a subject;
a top board moving device for moving the top board in a top board longitudinal direction;
a detector ring provided with a ring hole into which the top board is inserted in the top board longitudinal direction for detecting radiation emitted from inside of the subject;
a PET image acquisition device for acquiring a PET image as a sectional image showing radiopharmaceutical distribution in the subject based on detection data outputted from the detector ring; and
a CT image generation device provided with an introducing hole into which the top board is inserted in the top board longitudinal direction,
the apparatus further comprising:
a superimposing device for superimposing a CT image and the PET image, the detector ring and the CT image generation being arranged in the longitudinal direction,
the CT image generation device including:
a radiation source for emitting radiation;
a radiation detecting device for detecting radiation emitted from the radiation source;
a rotating device for synchronously rotates the radiation source and the radiation detecting device about the longitudinal direction as a center axis while maintaining a relative relationship therebetween; and
a CT image acquisition device for acquiring the CT image as a sectional image showing an internal structure of the subject based on detection data outputted from the radiation detecting device,
the top board moving device moving the top board in one direction along the longitudinal direction from an initial position to a termination position while stopping at given times, and simultaneously the detector ring and the radiation detecting device detecting radiation at every stop of the top board,
each image acquisition device acquiring each sectional image based on detection data outputted from the detector ring and the radiation detecting device when the top board is in each stop position, and
each of a first width and a second width being not less than one half of a center distance,
letting a distance between a first center and a second center be the center distance, the first center being a center of a range in the top board longitudinal direction where the detector ring acquires a PET image, and the second center being a center of a range in the top board longitudinal direction where the radiation detecting device acquires a CT image,
letting a width of a range in the top board longitudinal direction be the first width where the detector ring may acquire a PET image while the top board stops, and
letting a width of a range in the top board longitudinal direction be the second width where the CT image generation device may acquire a CT image while the top board stops.

2. (canceled)

3. The radiation tomography apparatus according to claim 1, wherein
each of the image acquisition devices acquires a sectional image throughout the subject by repeating acquisition of a sectional image for each section of the subject that is divided by every center distance in the top board longitudinal direction.

4. The radiation tomography apparatus according to claim 3, wherein
the top board moving device repeats a procedure of moving the top board in one direction by a length obtained by dividing a half length of the center distance by one or more integers and then stopping the to board.

5. The radiation tomography apparatus according to claim 4, wherein
the top board moving device repeats a procedure of moving the top board in one direction by half a length of the center distance and then stopping the top board.

6. The radiation tomography apparatus according to claim 1, wherein
a selecting device is provided to selectively execute one of (α) movement of the top board, (β) radiation detection by the detector ring, and (γ) radiation detection by the radiation detecting device, exclusively.

7. The radiation tomography apparatus according to claim 1, further comprising:
a period determination device for determining a period during which the subject moves; and
a synchronizing device for correlating the determined period with imaging,
each image acquisition device acquiring each sectional image only using detection data when movement of the subject lies in a phase.
